(19) United States
(12) Patent Application Publication
(10) Pub. No.: US 2017/0311916 A1
(43) Pub. Date: Nov. 2, 2017

(54) BLOOD-FLOW ANALYSIS DEVICE FOR BLOOD-FLOW SIMULATION, METHOD THEREFOR, AND COMPUTER SOFTWARE PROGRAM

(71) Applicant: EBM CORPORATION, Tokyo (JP)

(72) Inventors: Takanobu YAGI, Tokyo (JP); Young-Kwang PARK, Tokyo (JP)

(21) Appl. No.: 15/503,620

(22) PCT Filed: Oct. 8, 2015
(86) PCT No.: PCT/JP2015/078694

§ 371 Date: Jul. 18, 2017

Related U.S. Application Data
(60) Provisional application No. 62/061,435, filed on Oct. 8, 2014.

(51) Int. Cl. A61B 6/00; A61B 6/03; G06T 7/55; G06T 7/00

(52) U.S. Cl. CPC A61B 6/507; A61B 6/5217; G06T 7/0016; G06T 7/55; A61B 6/504; A61B 6/032; G06T 2207/30104

(57) ABSTRACT

The present method is a method for executing a computational fluid analysis on a blood flow in a computation object region, and displaying the analysis results, comprising the steps of: obtaining, by a computer, blood vessel shape data extracted from medical images; causing, by the computer, a user to specify a computation object region from the blood vessel shape data; retrieving, by the computer, a template according to the specified computation object region, wherein the template stores computation conditions validated for a blood flow analysis of the specified computation object region; and executing, by the computer, the computational fluid analysis of the blood flow in the computation object region by applying the computation conditions to the blood vessel shape data, and outputting the analysis results.

### FIELD OF THE INVENTION

The present invention relates to a blood flow analysis apparatus using computational fluid dynamics (Computational Fluid Dynamics, CFD). More specifically, it relates to a method for determining computation conditions, which is one of the data sets entered by a user when the blood flow analysis apparatus using CFD is utilized in medical settings.

### BACKGROUND OF THE INVENTION

In industrial fields in general, computational fluid dynamics (CFD) provides technology essential to design and development of automobiles, airplanes and the like. In industrial fields, CFD is normally implemented with so-called “general-purpose software.” The term “general-purpose” as in “general-purpose software” does not mean that the software may be used by “anyone,” but instead, it means that the software may be used for “any fluid” or “any flow.” In other words, the general-purpose software may be universally used for any fluid such as water, air, oil, etc. or for any flow such as laminar flow, transitional flow, turbulent flow, etc., but conditions used for each computation is determined by users, not developers of the software. Therefore, although the software is for “general purposes,” its users are typically experts with enough knowledge and expertise of CFD.

Such CFD-based blood flow simulations have been drawing attention since 2000's at the research level. On the other hand, the simulations' shortcomings have been also identified. The biggest challenge is the lack of commonality and standardization of CFD methodology due to its inherent dependence on users as described above.

This is also attributed to the fact that some of the users are medical doctors and technicians with no educational background in CFD. The CFD input includes four items: 1) flow channel shape, 2) fluid characteristics, 3) boundary conditions and 4) computation conditions. The computation conditions, one of the data sets the user enters into the blood flow analysis apparatus when using the apparatus in medical settings, include settings for computational grid generation, equation discretization and simultaneous equations solutions, all requiring general understanding of fluid dynamics; therefore, it is apparent that the commonality and standardization of CFD methodology do not advance when users without the required understanding use the apparatus.

Yet, when medical doctors or technicians without the CFD knowledge and experience use the apparatus for, for example, a blood flow simulation, it is unrealistic to demand that the users make appropriate judgment on the computation conditions.

Prior-art Reference 1: K. Zarins et al, Shear stress regulation of artery lumen diameter in experimental atherogenesis, J of VASCULAR SURGERY, 1985.

### SUMMARY OF THE INVENTION

In order to overcome the above challenges, according to a first principal aspect of the present invention, there is provided a method for executing a computational fluid analysis on a blood flow in a computation object region, and displaying an analysis result, comprising the steps of: obtaining, by a computer, blood vessel shape data extracted from medical images; causing, by the computer, a user to specify a computation object region from the blood vessel shape data; retrieving, by the computer, a template according to the specified computation object region, wherein the template stores computation conditions validated for a blood flow analysis of the specified computation object region; and executing, by the computer, a computational fluid analysis of the blood flow in the computation object region by applying the computation conditions to the blood vessel shape data, and outputting an analysis result.

According to one embodiment of the present invention, a plurality of the computation condition templates are prepared, wherein each computation condition template is prepared for each computation object region, and wherein the computation condition templates include templates for a cerebral artery, a carotid artery, a coronary artery and an aorta.

According to another embodiment, the computation condition templates stores conditions validated in advance by developers through comparisons with experiments, and comprise preset values which may not be changed by the user.

According to yet another embodiment, the computation condition templates further include prerequisites which vary depending on the specified computation object region.

In this case, the prerequisites preferably determine in advance whether or not non-Newtonian fluid characteristics and blood vessel wall mobility should be considered, respectively, for each computation object region.

Also, for the blood vessel wall mobility of the prerequisites, it is preferable that temporal shape changes of four-dimensional CTA data and the like are entered, and that a blood flow simulation using a moving boundary method is executed.

According to still another embodiment of the present invention, the above method further comprises the step of causing, by the computer, the user to specify one of computation precision levels with different computation time lengths, respectively.

In this case, it is preferable that the computation conditions included in the computation condition templates are a plurality of preset values corresponding to each computation precision level, and configured such that the user selects one of the plurality of preset values in the step of causing the user to specify one of the plurality of computation precision levels.

Also, it is preferable that one of the computation conditions included in the computation condition templates is a steady flow analysis, wherein a purpose of the computation condition is to analyze a flow field in a short period of time, and wherein the computation condition provides preset values based on an analysis technique prioritizing time rather than precision.

Further, it is preferable that one of the computation conditions included in the computation condition templates is a non-steady flow analysis, wherein the computation condition provides a plurality of preset values in controlling time and precision.

According to a second principal aspect of the present invention, there is provided a blood flow analysis apparatus for executing a computational fluid analysis on a blood flow in a computation object region, and displaying an analysis result, comprising: a computation object display section for obtaining, by a computer, blood vessel shape data extracted from medical images; a computation object region specifying section for causing, by the computer, a user to specify a computation object region from the blood vessel shape data; a blood flow analysis section for retrieving, by the computer, a template according to the specified computation object region, wherein the template stores computation conditions validated for a blood flow analysis of the specified computation object region, and executing, by the computer, a computational fluid analysis of a blood flow in the computation object region by applying the computation conditions to the blood vessel shape data; and a blood flow analysis results output section for outputting an analysis result by the computer.

According to a third principal aspect of the present invention, there is provided a computer software program for executing a computational fluid analysis on a blood flow in a computation object region, and displaying an analysis result, said computer software program comprising instructions for executing the steps of: obtaining blood vessel shape data extracted from medical images; causing a user to specify a computation object region from the blood vessel shape data; retrieving, by the computer, a template according to the specified computation object region, wherein the template stores computation conditions validated for a blood flow analysis of the specified computation object region; and executing a computational fluid analysis of the blood flow in the computation object region by applying the computation conditions to the blood vessel shape data, and outputting an analysis result.

The above and other characteristics of the present invention will be readily appreciated by those skilled in the art by referring to the following Detailed Description of the Invention and the accompanying drawings.

### BRIEF DESCRIPTION OF THE DRAWINGS

FIG. 1 is a diagram describing computational fluid dynamics and computation conditions;

FIG. 2 is a diagram showing a flow of blood flow analysis using computational fluid dynamics;

FIG. 3(a) is a diagram showing shear stress vectors on a brain aneurysm using upwind differencing with first-order precision, and FIG. 3(b) shows the same using second-order precision;

FIG. 4 is a schematic structural view showing one embodiment of the present invention;

FIG. 5 is a diagram showing an input interface of the present embodiment;

FIG. 6 is a diagram showing an example of computation condition template of the present embodiment;

FIG. 7 is a diagram showing an example of preset values of computation conditions of the present embodiment;

FIG. 8 is a diagram showing an example of computational grid generation in the present embodiment; and

FIG. 9 is a diagram showing an example of validation of a computation condition in the present embodiment.

### DETAILED DESCRIPTION OF THE INVENTION

One embodiment of the present invention will be described specifically below in accordance with accompanying drawings.

The present invention relates to a blood flow analysis device 1 for a blood flow analysis using computational fluid dynamics (Computational Fluid Dynamics, CFD). In particular, the present device validates computation conditions as one of the data sets entered for the blood flow analysis by comparing experimental and computed values for each object blood vessel region, and provides the validated information as a user-uneditable preset template to thereby enable users such as physicians unfamiliar with CFD to perform an appropriate blood flow simulation.

First, overview of processing using CFD will be discussed below in order to simplify the discussion of the present embodiment.

### (Computational Fluid Analysis Processing)

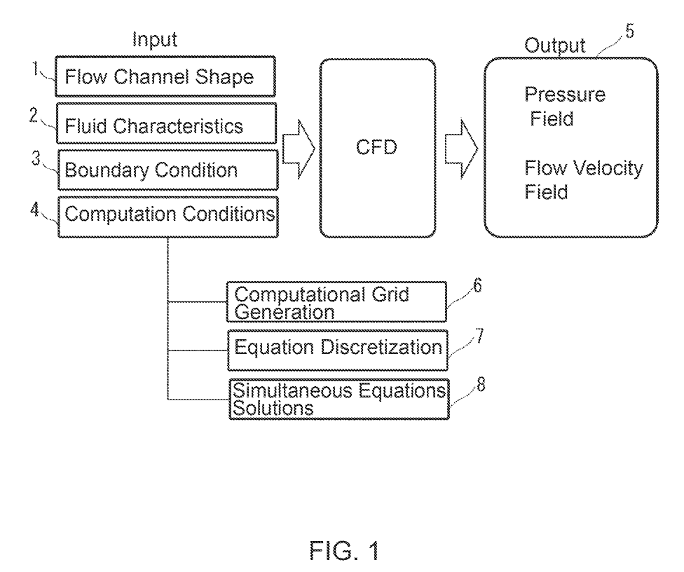

Computational fluid dynamics (CFD) provides technologies for determining a fluidic flow using a computational analysis. As shown in FIG. 1, the present example uses a flow channel shape 1, fluid characteristics 2, a boundary condition 3 and computation conditions 4 as input data. Based on these input items, CFD operations are performed to output pressure and flow velocity fields 5 in a blood flow space. In this example, CFD operations are executed using the time evolution concept to obtain the time-space pressure and flow velocity fields 5.

Here, the flow channel shape 1 discussed above is constructed by processing medical images and extracting a blood vessel shape, or by designing a blood vessel shape with CAD (computer-aided design) and the like on a computer. The fluid characteristics 2 in this example are density and viscosity. The boundary condition 3 is specifically flow velocity and pressure distributions at an end face of each conduit line, and a constraint condition at a wall surface. For example, as for the flow velocity distribution at an inlet or an outlet of a conduit line, the fluid slip is ignored and the flow velocity is set to zero at the wall surface (no-slip condition). The computation conditions 4, which are the subject matter of the present invention, include computational grid generation 6, equation discretization 7 regarding equations solutions and simultaneous equations solutions 8 for a given flow channel shape 1.

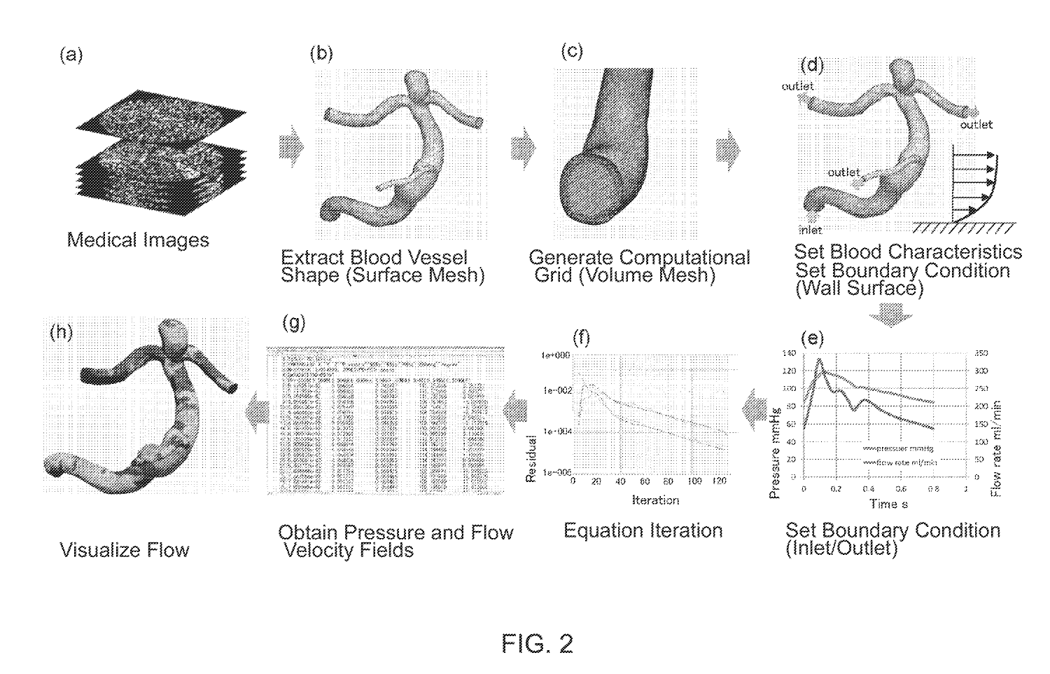

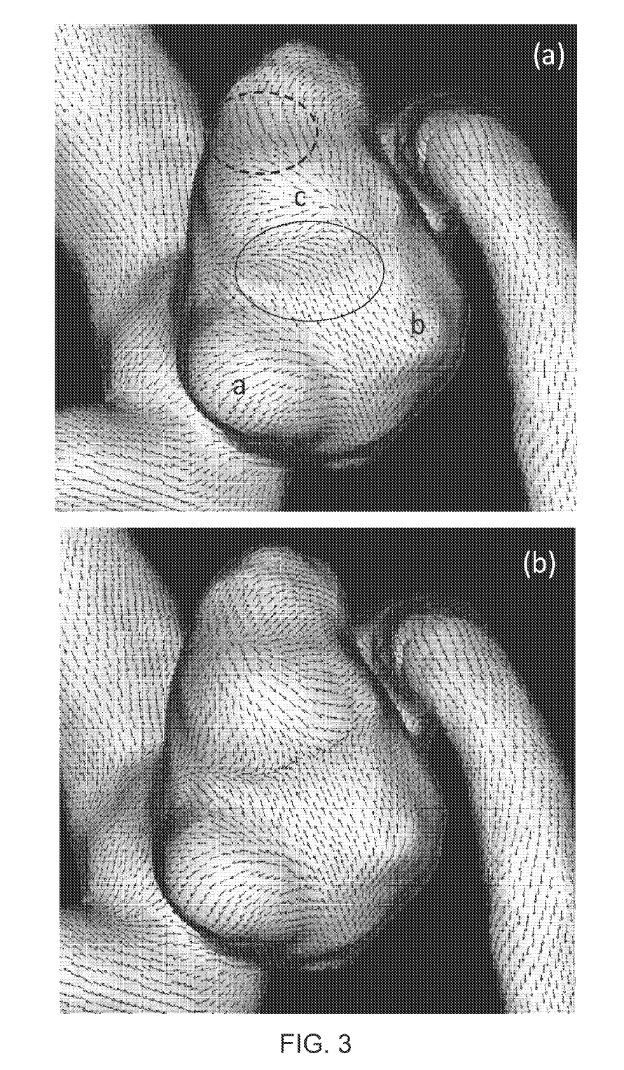

Next, the computational grid generation 6, the equation discretization 7 and the simultaneous equations solutions 8 of the computation conditions 4 will be described with reference to FIG. 2, showing a blood flow analysis flow, and FIG. 3.

### (Computational Grid Generation 6)

Computational grids are generated in steps shown in FIG. 2(c), but first in (b), a flow channel shape 1 is constructed based on medical images (a). Here, the computational grids are generated to make up a volume mesh from fine elements of an interior of a flow channel shape (b) provided as a surface mesh. The computational grids are determined by taking into account: 1) size, 2) shape, 3) density, 4) distribution, 5) orientation and the like.

### (Equation Discretization 7 and Simultaneous Equations Solutions 8)

Next, overview of the equation discretization 7 and the simultaneous equations solutions 8 will be discussed below with respect to Equation 1.

$$\begin{matrix}Continuous \\ equation\end{matrix} \frac{\partial u}{\partial x} + \frac{\partial v}{\partial y} + \frac{\partial w}{\partial z} = 0 ρ ( \frac{\partial u}{\partial t} + u \frac{\partial u}{\partial x} + v \frac{\partial u}{\partial y} + w \frac{\partial u}{\partial z} ) = - \frac{\partial p}{\partial x} + µ ( \frac{{\partial }^{2} u}{\partial {x}^{2}} + \frac{{\partial }^{2} u}{\partial {y}^{2}} + \frac{{\partial }^{2} u}{\partial {z}^{2}} ) + ρ {f}_{x} \\ \begin{matrix}Navier \text{-} Stokes \\ equations\end{matrix} ρ ( \frac{\partial v}{\partial t} + u \frac{\partial v}{\partial x} + v \frac{\partial v}{\partial y} + w \frac{\partial v}{\partial z} ) = - \frac{\partial p}{\partial y} + µ ( \frac{{\partial }^{2} v}{\partial {x}^{2}} + \frac{{\partial }^{2} v}{\partial {y}^{2}} + \frac{{\partial }^{2} v}{\partial {z}^{2}} ) + ρ {f}_{y} ρ ( \frac{\partial w}{\partial t} + u \frac{\partial w}{\partial x} + v \frac{\partial w}{\partial y} + w \frac{\partial w}{\partial z} ) = - \frac{\partial p}{\partial z} + µ ( \frac{{\partial }^{2} w}{\partial {x}^{2}} + \frac{{\partial }^{2} w}{\partial {y}^{2}} + \frac{{\partial }^{2} w}{\partial {z}^{2}} ) + ρ {f}_{z} \\ ρ \underbrace{( \frac{\partial u}{\partial t}}_{{Temporal}_{acceleration}} + \underbrace{u \frac{\partial u}{\partial x} + v \frac{\partial u}{\partial y} + w \frac{\partial u}{\partial z} )}_{{Advection}_{acceleration}} = { \underbrace{- \frac{\partial p}{\partial x} +}_{{Pressure}_{- dependent term} \\} }_{} \underbrace{µ ( \frac{{\partial }^{2} u}{\partial {x}^{2}} + \frac{{\partial }^{2} u}{\partial {y}^{2}} + \frac{{\partial }^{2} u}{\partial {z}^{2}} )}_{{Viscosity}_{- dependent term}} \underbrace{+ ρ {f}_{x}}_{{External force }_{- dependent term} } \qquad [ Equation 1 ]$$

In equation discretization, a differential equation is replaced with an algebraic equation. Navier-Stokes equations are nonlinear second-order differential equations and their exact solutions have not been obtained mathematically. For this reason, the differential equations are replaced with algebraic equations by discretizing each element constituting the differential equations. The simultaneous equations solutions are ways to simultaneously establishing continuous equations and the Navier-Stokes equations.

The computational grid generation 6, the equation discretization 7 and the simultaneous equations solutions 8 of the computation conditions discussed above have the following difficulties in terms of setting the computation conditions.

That is, first in the computational grid generation 6, although it has been discussed above that the computational grids are determined by taking into account 1) size, 2) shape, 3) density, 4) distribution, 5) orientation and the like, the flow needs to be treated differently in the bulk stream and in the boundary layer near the wall, requiring finer computational grids for regions with high velocity gradients as in the boundary layer. Discontinuity and distortion of the computational grids may compromise the convergence and precision of computation. There are some computational grid types including the prism, tetrahedron and hexahedron. Overly fine computational grids may lead to a pointlessness increase of computation time. Thurs, the computational grid needs to be carefully configured between the time and precision requirements. There is no agreed-upon standard for how to determine computational grids, and other conditions are determined by identifying the degree of computational-grid dependency with comparative tests and selecting the condition with the least dependency. Sometimes, the nature of flow such as a laminar flow or turbulence needs to be considered. In the case of turbulence, the computational grids are typically arranged to allow enough image resolution to identify a thin layer called “viscous sublayer” in a boundary layer. Generating the computational grids requires general understanding of fluid dynamics, and it is difficult to generate the grids for users such as physicians unfamiliar with CFD.

Further, in the equation discretization 7 and the simultaneous equations solutions 8, the Navier-Stokes equations are nonlinear second-order differential equations and their exact solutions cannot be obtained mathematically as discussed above. Accordingly, the differential equations are replaced with algebraic equations by discretizing each element constituting the differential equations.

$$\begin{matrix}Continuous \\ equation\end{matrix} \frac{\partial u}{\partial x} + \frac{\partial v}{\partial y} + \frac{\partial w}{\partial z} = 0 ρ ( \frac{\partial u}{\partial t} + u \frac{\partial u}{\partial x} + v \frac{\partial u}{\partial y} + w \frac{\partial u}{\partial z} ) = - \frac{\partial p}{\partial x} + µ ( \frac{{\partial }^{2} u}{\partial {x}^{2}} + \frac{{\partial }^{2} u}{\partial {y}^{2}} + \frac{{\partial }^{2} u}{\partial {z}^{2}} ) + ρ {f}_{x} \\ \begin{matrix}Navier \text{-} Stokes \\ equations\end{matrix} ρ ( \frac{\partial v}{\partial t} + u \frac{\partial v}{\partial x} + v \frac{\partial v}{\partial y} + w \frac{\partial v}{\partial z} ) = - \frac{\partial p}{\partial y} + µ ( \frac{{\partial }^{2} v}{\partial {x}^{2}} + \frac{{\partial }^{2} v}{\partial {y}^{2}} + \frac{{\partial }^{2} v}{\partial {z}^{2}} ) + ρ {f}_{y} ρ ( \frac{\partial w}{\partial t} + u \frac{\partial w}{\partial x} + v \frac{\partial w}{\partial y} + w \frac{\partial w}{\partial z} ) = - \frac{\partial p}{\partial z} + µ ( \frac{{\partial }^{2} w}{\partial {x}^{2}} + \frac{{\partial }^{2} w}{\partial {y}^{2}} + \frac{{\partial }^{2} w}{\partial {z}^{2}} ) + ρ {f}_{z} \\ ρ \underbrace{( \frac{\partial u}{\partial t}}_{{Temporal}_{acceleration}} + \underbrace{u \frac{\partial u}{\partial x} + v \frac{\partial u}{\partial y} + w \frac{\partial u}{\partial z} )}_{{Advection}_{acceleration}} = \underbrace{- \frac{\partial p}{\partial x} +}_{{Pressure}_{- dependent term}} \underbrace{µ ( \frac{{\partial }^{2} u}{\partial {x}^{2}} + \frac{{\partial }^{2} u}{\partial {y}^{2}} + \frac{{\partial }^{2} u}{\partial {z}^{2}} )}_{{Viscosity}_{- dependent term}} \underbrace{+ ρ {f}_{x}}_{{External force}_{- dependent term}}$$

In general, each term of the Navier-Stokes equations are treated differently. In particular, the terms of temporal acceleration and advection acceleration are important. Discretization of the temporal acceleration may be performed using the first- and second-order backward Euler methods, etc. For unsteady computations, time steps are specified. When solving a highly unsteady flow with the explicit method, Δt is determined so that the Courant number c=u Δt/Δx is less than 1. Here, u is the velocity and Δx is the grid size. For the implicit method, the Courant number does not have to be less than 1, but if the number is overly large, it may cause divergence. Among other factors, the discretization of advection acceleration has the greatest impact on the analysis results. The advection acceleration contributes to the flow nonlinearity and has a strong influence on the precision and convergence of the results. The upwind differencing is often used for discretizing the advection acceleration, but selection between the first- and second-order accuracies of the upwind differencing scheme must be made by considering numeric viscosity and convergence, requiring high expertise. The simultaneous equations solutions are ways to simultaneously establishing continuous equations and the Navier-Stokes equations, and have a plurality of techniques, similarly requiring high expertise. Thus, it is difficult for users such as physicians unfamiliar with CFD to conduct appropriate blood flow simulations.

As for the high expertise required in setting the computation conditions, FIGS. 3(a) and (b) specifically illustrate the discretization of advection acceleration, which affects the analysis results the most. In FIGS. 3(a) and (b), differences of shear stress vectors on a brain aneurysm caused by differences in advection acceleration (in these figures, shear stress vectors are displayed in unit vectors). FIGS. 3(a) and (b) show discretization of advection acceleration by upwind differencing with first-order precision and second-order precision, respectively. All other condition factors are the same in both figures. The blood flows from the lower depth towards the viewer on the line of sight, and flows between blebs a, b to a bleb c. Before and after the bleb c, different flows are seen in the two figures. With the first-order precision, the flow is smoothed by the numeric viscosity, but with the second-order precision, merging and collisions of the flow near the bleb are successfully reproduced.

In the present invention, each setting of the computation conditions requiring high expertise as described above may be performed by validating the computation conditions based on the comparison between experimental and computed values for each object blood vessel region, and providing the validated computation conditions as in the user-uneditable preset template to thereby enable users such as physicians unfamiliar with CFD to perform an appropriate blood flow simulation.

The detailed description will be provided below.

### Embodiment of the Present Invention

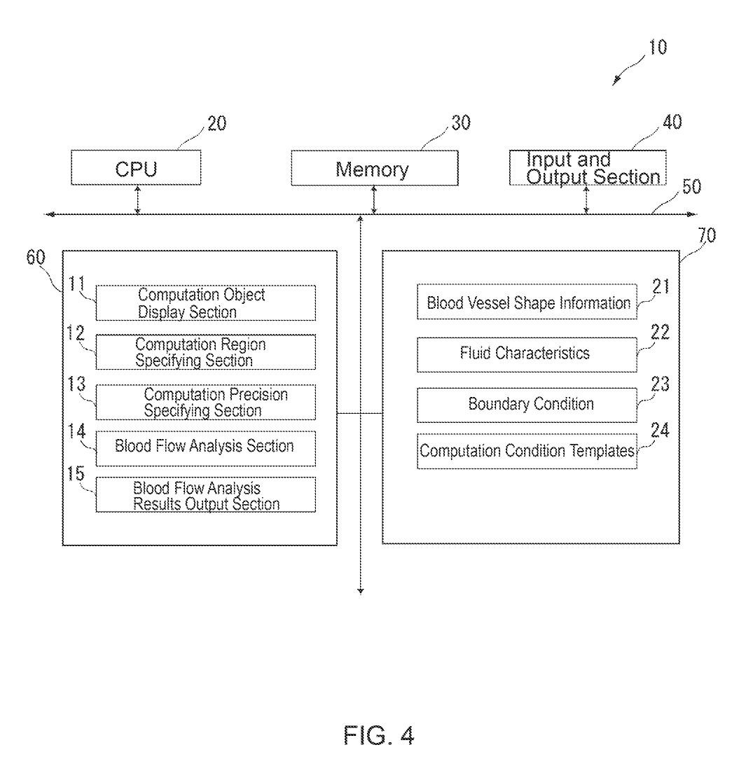

FIG. 4 is a schematic structural view showing a blood flow analysis device according to the present embodiment.

The blood flow analysis device 10 is defined by a CPU 20, a memory 30 and an input and output section 40, which are connected with a bus 50, which in turn is connected with a program storage section 60 and a data storage section 70 for storing data.

The program storage section 60 is equipped with a computation object display section 11, a computation region specifying section 12, a computation precision specifying section 13, a blood flow analysis section 14 and a blood flow analysis results output section 15. The data storage section 70 is equipped with a blood vessel shape information 21, a fluid characteristics 22, a boundary condition 23 and a computation condition template 24.

The above structural requirements (the computation object display section 11, the computation region specifying section 12, computation precision specifying section 13, blood flow analysis section 14 and blood flow analysis section 15) are configured with computer software stored in a storage area of a hard disk, called by the CPU 20, and deployed and executed on the memory 30 to thereby serve as respective components of the present invention.

### Example of Input Interface

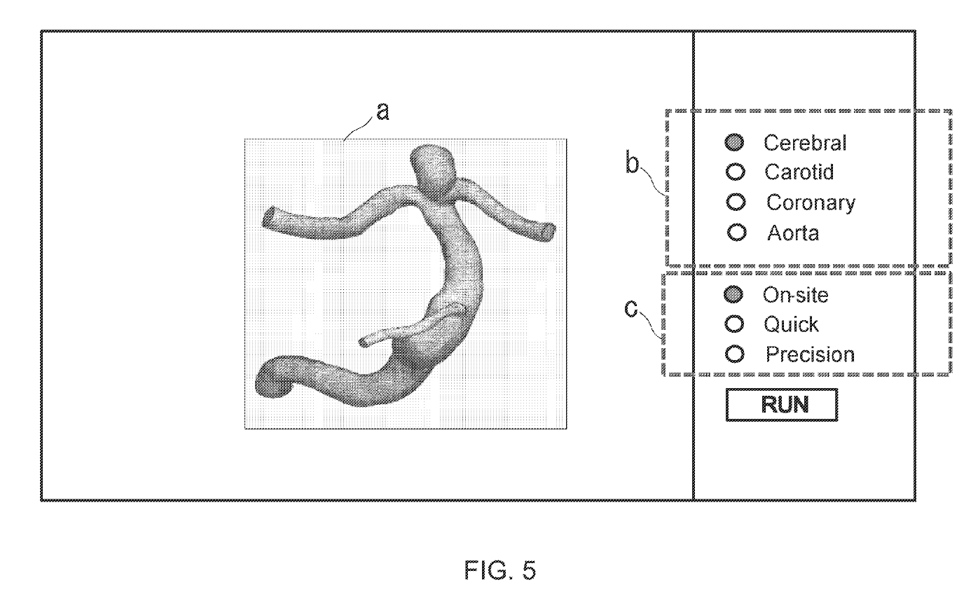

Next, an input interface of software dedicated to blood flow analyses according to the present embodiment will be described below in reference to FIG. 5.

This input interface comprises an area a displayed by the computation object display section 11, an area b displayed by the computation region specifying section 12 and an area c displayed by the computation precision specifying section 13. In the area a displayed by the computation object display section 11, a blood vessel shape extracted from medical images are retrieved from the blood vessel shape information section 21 and displayed. In the area b displayed by the computation region specifying section 12, a computation object display section (cerebral artery (Cerebral), carotid artery (Carotid), coronary artery (Coronary) or aorta (Aorta)) is displayed so that the user may make a selection. In the area c displayed by the computation precision specifying section 13, On-site (about 10 minutes), Quick (about 2 hours) and Precision (about 1 day) are displayed so that the computation conditions may be selected, taking account of the balance between analysis precision and time required. If the user specifies a computation object region in the area (b) and a computation precision in the area (c), the blood flow analysis section 14 retrieves computation conditions corresponding with the user specification from the computation condition template 24. The blood flow analysis section 14 applies the computation conditions to the blood vessel shape data of the computation object region displayed in the area a to thereby perform a blood flow analysis using CFD. The results of the blood flow analysis performed by the blood flow analysis section 14 are output by the blood flow analysis results output section 15. Thus, the user only needs to specify the computation object region and the computation precision, and a computer may extract a computation condition template optimal for each condition from information stored in the memory to calculate CFD.

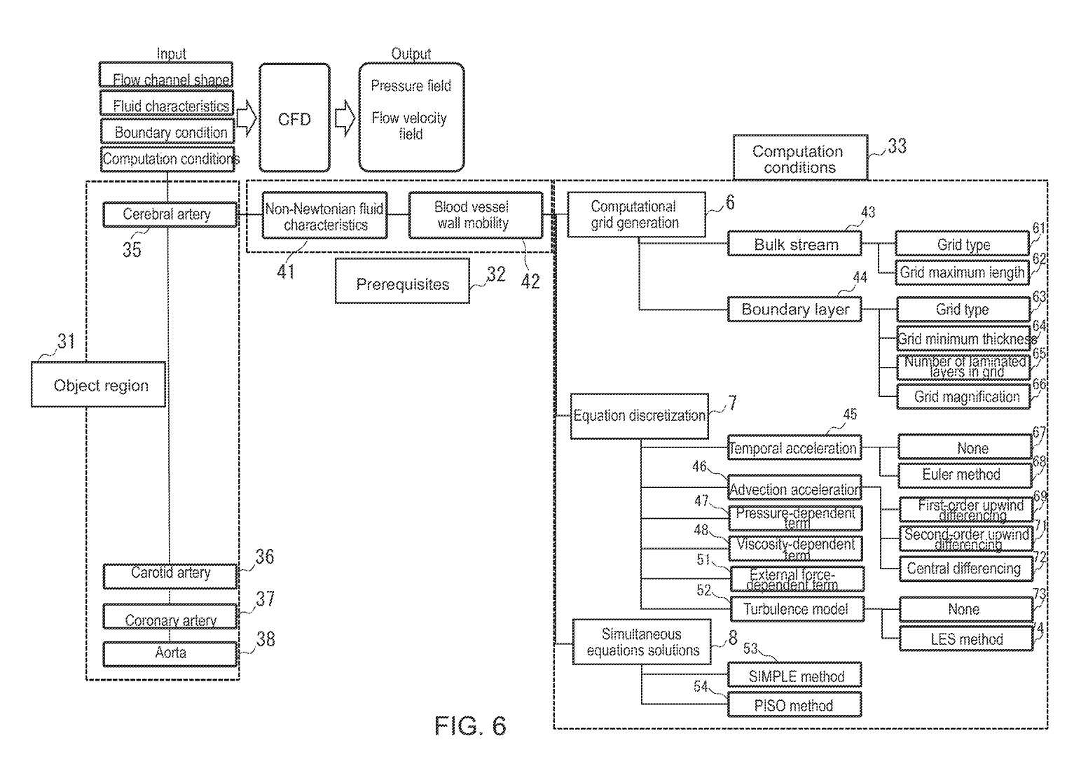

FIG. 6 shows a structure of a computation condition template of the present embodiment. Each condition value stored in this computation condition template is given as a preset value or a preset condition which may not be changed by the user.

This computation condition template comprises a three-stage structure made of an object region 31, prerequisites 32 and computation conditions 33.

In this example, 1) object region 31 is, for example, a cerebral artery 35, a carotid artery 36, a coronary artery 37 or an aorta 38. The prerequisites 32 and the computation conditions 33 are preset for each of these object regions, but in the example of FIG. 6, only one example of cerebral artery is shown.

2) The prerequisites 32 vary depending on the object region type, but in this example of cerebral artery 35, non-Newtonian fluid characteristics 41 and blood vessel wall mobility 42 are included. The non-Newtonian fluid characteristics 41 is information on whether or not the blood viscosity should be of a type dependent on the shear velocity at a location in question. If the non-Newtonian fluid characteristics 41 is not of the dependent type, a constant value will be used. If the dependent type is selected, an iteration loop of computation is added. The blood vessel wall mobility 42 (presence or absence of) is selected for regions with a significant change in blood vessel shape such as an aorta. It has been validated that the shape change does not need to be considered for cerebral arteries. In the present embodiment, the prerequisites 32 are automatically determined when the object region 31 is determined.

3) The computation conditions 33 include respective conditions of the computational grid generation 6, the equation discretization 7 and the simultaneous equations solutions 8.

In this example, a mainstream 43 and a boundary layer 44 are included as conditions for the computational grid generation 6. The mainstream 43 further includes conditions: a grid type 61 and a grid maximum length 62.

Conditions of the equation discretization 7 include a temporal acceleration 45, an advection acceleration 46, a pressure-dependent term 47, a viscosity-dependent term 48, an external force-dependent term 51 and a turbulence model 52. The temporal acceleration 45 further includes “none” 67 and an Euler method 68. The advection acceleration 46 further includes a first-order upwind differencing 69, a second-order upwind differencing 71 and a central differencing 72. The turbulence model 52 further includes “none” 73 and a LES method 74.

Conditions of the simultaneous equations solutions 8 include a SIMPLE method 53 and a PISO method 54.

Further, in the present embodiment, a plurality of patterns are prepared as respective values of the above computation conditions 33 according to the computation time required, i.e., the On-site 81 (about 10 minutes), the Quick 82 (about 2 hours) and the Precision 83 (about 1 day). In other words, the user will first select the object region 31, and then, the desirable computation time.

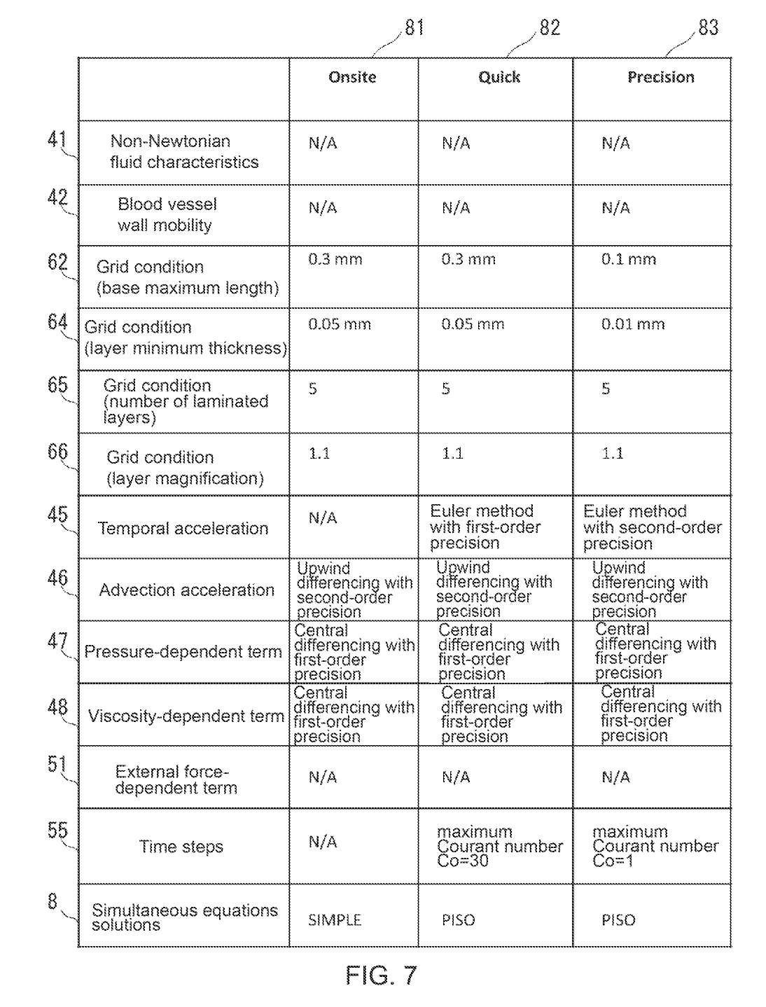

FIG. 7 shows an example of preset value templates of the computation conditions 6-74 for each of the On-site 81 (about 10 minutes), the Quick 82 (about 2 hours) and the Precision 83 (about 1 day).

Here, the On-site 81 has a computation condition template which does not consider the temporal acceleration 45 in the equation discretization 7. Whereas, the Quick 82 and the Precision 83 have computation condition templates considering the temporal acceleration 45.

Other than the temporal acceleration 45, the non-Newtonian fluid characteristics 41, the blood vessel wall mobility 42, the grid condition (base maximum length) 62, the grid condition (layer minimum thickness) 64, the grid condition (the number of laminated layers) 65, the grid condition (layer magnification) 66, the advection acceleration 46, the pressure-dependent term 47, the viscosity-dependent term 48, the external force-dependent term 51, time steps 55 and the simultaneous equations solutions 8 are set as preset values or preset conditions of the computation conditions for the On-site 81, the Quick 82 and the Precision 83. In this example, the non-Newtonian fluid characteristics 41, the blood vessel wall mobility 42 and the external force-dependent term 51 are not considered for any computation time length, but the other computation conditions are respectively configured as illustrated.

Here, each value of the prepared computation conditions are ones already validated (the computational grid generation, the equation discretization and the simultaneous equations solutions indicated by 6, 7 and 8 in FIG. 6, respectively). Now, validation steps will be described.

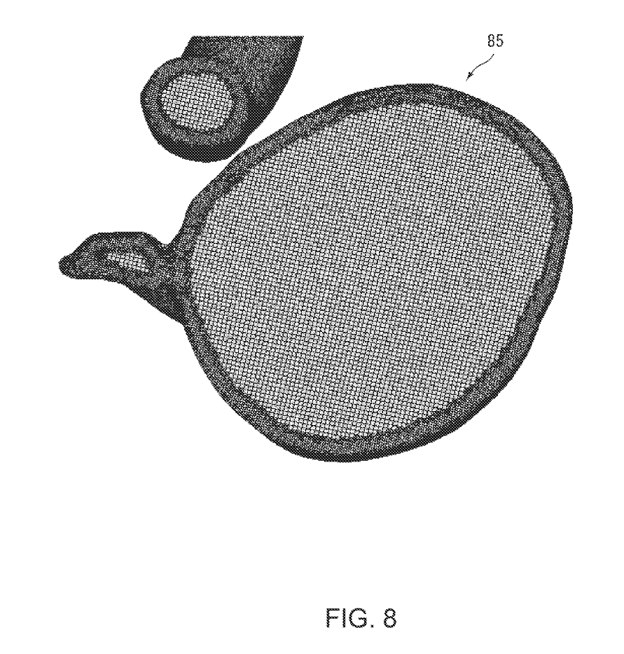

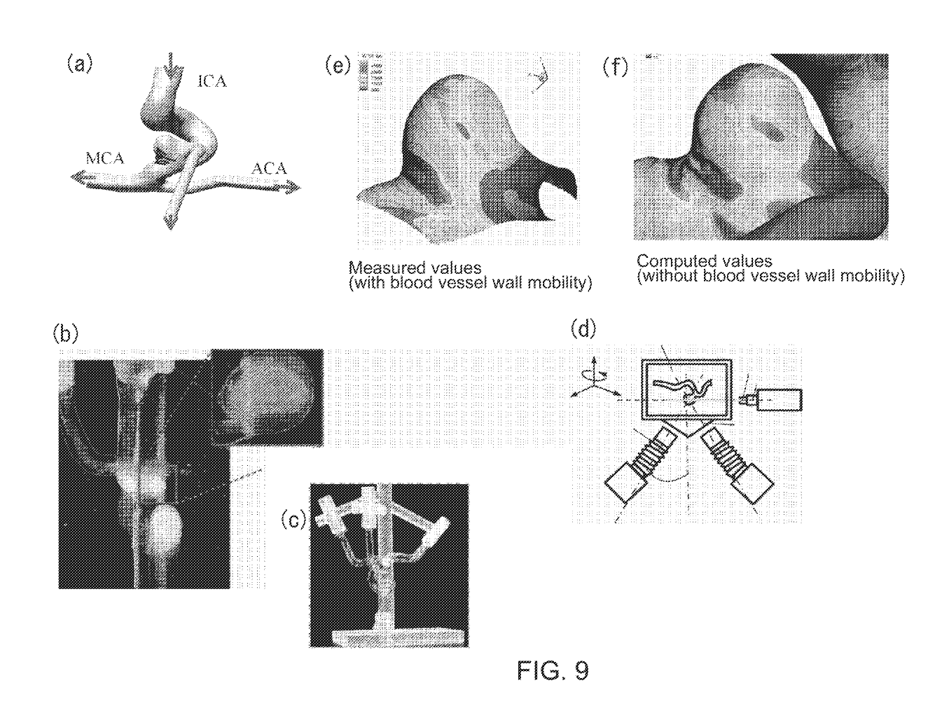

Firstly, FIG. 8 shows an example of computational grid 85 generated with a cerebral artery as the object. Based on this, validation is performed with the steps shown in FIG. 9.

In this example, comparison with an experimental solution is shown as one of the methods for validating the computation conditions.

There are two types of experiments: in vivo and in vitro. For in vivo experiments, computed values of the flow velocity may be compared with measured values obtained by, for example, the phase-contrast MRI method. In vitro experiments were performed by building an in vitro blood vessel model as shown in FIG. 9(c) based on the constructed blood vessel model (FIG. 9(a)), and measuring the flow velocity in a reconstructed flow field with good reproducibility using, for example, the particle image velocimetry (PIV). These in vitro experiments are effective since in vivo experiments has a resolution limit of 0.5-1.0 mm and are unable to yield important metrics, such as a wall surface shear stress, with good precision.

Thus, in this example, the fluid velocity was measured with the spatial resolution of 0.1 mm in the in vitro experiments (J. R. Soc. Interface, 2013 10, T. Yagi, et al.). The PIV method is shown in FIG. 9(d). In other words, a blood-mimicking material was seeded with fluorescent particles as flow tracer particles. Displacement of each particle was measured with two cameras to obtain three components of the particle's velocity. By doing this in multiple cross-sections, a three-dimensional structure of the flow field was measured (FIG. 9(b)).

Using the wall surface shear stress computed from such experiments, FIGS. 9(e) and (f) show the comparison between the experimental and computed solutions, respectively. The computed solution is based on the preset values set in the templates described above. Thurs, well-matched values between the experiment and computation are used as validated preset values.

Note that in experiments, the blood vessel wall mobility of elastic walls is taken into account. In the computation, rigid walls are considered. In both cases, the Newtonian fluid is used. This good match between the experimental and computed solutions show that the blood vessel wall mobility does not need to be considered in the cerebral artery region. Using the preset computation conditions validated one by one as above is one of the characteristics of the present invention.

As described above, the present invention limits object regions to only blood flows and further limits the object blood vessels to thereby provide dedicated software verified and validated by the developers. Also, the present invention provides a blood flow analysis apparatus for storing the detailed preset computation conditions in a memory and loading the preset computation conditions to perform computations, wherein the preset computation conditions were determined by the developers during their development stage as optimal values for the computation conditions by comparing with experimental solutions. More specifically, the computation condition templates were made possible by limiting the scope of the CFD application (cerebral arteries, carotid arteries, coronary arteries and aortas, etc.). In this manner, the blood flow analysis apparatus capable of automatically setting the validated computation conditions well-adapted to onsite environment may be provided to users such as medical doctors or technicians without the CFD knowledge and experience. Also, unlike in the industrial fields, in the medical field, where the trade-off between the time and precision has high stakes, the blood flow analysis apparatus of the present invention may provide computation conditions satisfying the required precision within a limited time.

Needless to say, the present invention may be modified in various manners and is not limited to the above one embodiment, and various changes and modifications may be made without departing from the scope and spirit of the invention.

1. A method for executing a computational fluid analysis on a blood flow in a computation object region, and displaying analysis results, comprising the steps of:
obtaining, by a computer, blood vessel shape data extracted from medical images;
causing, by the computer, a user to specify a computation object region from the blood vessel shape data;
retrieving, by the computer, a template according to the specified computation object region, wherein the template stores computation conditions validated for a blood flow analysis of the specified computation object region; and
executing, by the computer, a computational fluid analysis of a blood flow in the computation object region by applying the computation conditions to the blood vessel shape data, and outputting an analysis result.

2. The method of claim 1, wherein
a plurality of computation condition templates are prepared, wherein each computation condition template is produced for each computation object region, and wherein the computation condition templates include templates for a cerebral artery, a carotid artery, a coronary artery and an aorta.

3. The method of claim 1, wherein
the computation condition template stores conditions validated in advance by developers through comparisons with experiments, and comprises preset values which may not be changed by the user.

4. The method of claim 1, wherein
the computation condition template further include prerequisites which vary depending on the specified computation object region.

5. The method of claim 4, wherein
the prerequisites determine in advance whether or not non-Newtonian fluid characteristics and blood vessel wall mobility should be considered, respectively, for each computation object region.

6. The method of claim 5, wherein
for the blood vessel wall mobility of the prerequisites, temporal shape changes of four-dimensional CTA data and the like are entered, and a blood flow simulation using a moving boundary method is executed.

7. The method of claim 1, further comprising the step of:
causing, by the computer, the user to specify one of computation precision levels having different respective computation time lengths.

8. The method of claim 7, wherein
the computation conditions included in the computation condition template are a plurality of preset values corresponding to each computation precision level, and configured such that the user selects one of the plurality of preset values in the step of causing the user to specify one of the plurality of computation precision levels.

9. The method of claim 8, wherein
one of the computation conditions included in the computation condition templates is a steady flow analysis, wherein a purpose of the computation condition is to analyze a flow field in a short period of time, and wherein the computation condition provides preset values based on an analysis technique prioritizing time rather than precision.

10. The method of claim 8, wherein
one of the computation conditions included in the computation condition template is a non-steady flow analysis, wherein the computation condition provides a plurality of preset values in controlling time and precision.

11. A blood flow analysis apparatus for executing a computational fluid analysis on a blood flow in a computation object region, and displaying analysis results, comprising:
a computation object display section for obtaining, by a computer, blood vessel shape data extracted from medical images;
a computation object region specifying section for causing, by the computer, a user to specify a computation object region from the blood vessel shape data;
a blood flow analysis section for retrieving, by the computer, a template according to the specified computation object region, wherein the template stores computation conditions validated for a blood flow analysis of the specified computation object region, and executing, by the computer, a computational fluid analysis of a blood flow in the computation object region by applying the computation conditions to the blood vessel shape data; and
a blood flow analysis results output section for outputting an analysis result by the computer.

12. The blood flow analysis apparatus of claim 11, wherein
a plurality of computation condition templates are prepared, wherein each computation condition template is prepared for each computation object region, and wherein the computation condition templates include templates for a cerebral artery, a carotid artery, a coronary artery and an aorta.

13. The blood flow analysis apparatus of claim 11, wherein
the computation condition template stores conditions validated in advance by developers through comparisons with experiments, and comprises preset values which may not be changed by the user.

14. The blood flow analysis apparatus of claim 11, wherein
the computation condition template further includes prerequisites which vary depending on the specified computation object region.

15. The blood flow analysis apparatus of claim 14, wherein
the prerequisites determine in advance whether or not non-Newtonian fluid characteristics and blood vessel wall mobility should be considered, respectively, for each computation object region.

16. The blood flow analysis apparatus of claim 15, wherein
for the blood vessel wall mobility of the prerequisites, temporal shape changes of four-dimensional CTA data and the like are entered, and a blood flow simulation using a moving boundary method is executed.

17. The blood flow analysis apparatus of claim 11, further comprising:
a computation precision specifying section for causing, by the computer, the user to specify one of computation precision levels having different respective computation time lengths.

18. The blood flow analysis apparatus of claim 17, wherein
the computation conditions included in the computation condition template are a plurality of preset values corresponding to each computation precision level, and configured such that the user selects one of the plurality of preset values using the computation precision specifying section.

19. The blood flow analysis apparatus of claim 18, wherein
one of the computation conditions included in the computation condition template is a steady flow analysis, wherein a purpose of the computation condition is to analyze a flow field in a short period of time, and wherein the computation condition provides preset values based on an analysis technique prioritizing time rather than precision.

20. The blood flow analysis apparatus of claim 18, wherein
one of the computation conditions included in the computation condition template is a non-steady flow analysis, wherein the computation condition provides a plurality of preset values in controlling time and precision.

21-30. (canceled)
